(19) United States
(12) Patent Application Publication
(10) Pub. No.: US 2023/0157637 A1
(43) Pub. Date: May 25, 2023

(54) INTRAORAL ELECTRONIC SENSING FOR HEALTH MONITORING

(71) Applicant: Augmental Technologies Inc., San Francisco, CA (US)

(72) Inventors: Tomas Alfonso Vega Galvez, San Francisco, CA (US); Corten Singer, San Francisco, CA (US)

(73) Assignee: Augmental Technologies Inc., San Francisco, CA (US)

(21) Appl. No.: 18/099,288

(22) Filed: Jan. 20, 2023

Related U.S. Application Data
(63) Continuation-in-part of application No. 17/366,186, filed on Jul. 2, 2021.
(60) Provisional application No. 63/301,501, filed on Jan. 21, 2022.
(60) Provisional application No. 63/162,444, filed on Mar. 17, 2021.
(60) Provisional application No. 63/063,455, filed on Aug. 10, 2020.
(60) Provisional application No. 63/047,946, filed on Jul. 3, 2020.

(51) Int. Cl. A61B 5/00; A61B 5/0205; A61B 5/11; A61B 5/293

(52) U.S. Cl. CPC A61B 5/682; A61B 5/0022; A61B 5/02055; A61B 5/1118; A61B 5/293; A61B 5/4812; A61B 5/02427

(57) ABSTRACT

Intraoral electronic sensing for health monitoring is disclosed. Wireless connectivity is provided between a processor and a wireless transmitting device. The wireless transmitting device is embedded in an intraoral sensing interface for use in a person. Sensors are coupled to the wireless transmitting device, wherein the sensors are attached to the intraoral sensing interface. The sensors include a photoplethysmography (PPG) sensor to detect cardiac activity, a breathing sensor to detect pulmonary function, an inertial measurement unit (IMU) sensor to detect three-dimensional motion, and a temperature sensor to monitor body temperature. Further sensors include an electroencephalogram sensor to detect brain activity. Health data about the person is provided to a receiving device, based on data from one or more of the PPG sensor, the breathing sensor, the IMU sensor, and the temperature sensor. The health data is provided using the wireless connectivity.

### RELATED APPLICATIONS

This application claims the benefit of U.S. provisional patent application “Intraoral Electronic Sensing for Health Monitoring” Ser. No. 63/301,501, filed Jan. 21, 2022.

This application is also a continuation-in-part of U.S. patent application “Data Manipulation Using Remote Augmented Sensing” Ser. No. 17/366,186, filed Jul. 2, 2021, which claims the benefit of U.S. provisional patent applications “Data Manipulation Using Remote Augmented Sensing” Ser. No. 63/047,946, filed Jul. 3, 2020, “Gestural Sensing Using In-Ear Inertial Measurements” Ser. No. 63/063,455, filed Aug. 10, 2020, and “Intraoral Connected Processing Devices” Ser. No. 63/162,444, filed Mar. 17, 2021.

Each of the foregoing applications is hereby incorporated by reference in its entirety.

### FIELD OF ART

This application relates generally to health monitoring and more particularly to health monitoring using intraoral electronic sensing.

### BACKGROUND

The variety of electronic devices or “gadgets” that are available today is simply staggering. While some of these devices are designed for learning, work, research, business management, government services, and facilities operations, among many additional uses, other devices are designed for diversion, entertainment, and generally, for fun. The electronic devices can range from desktop computers and laptop computers to tablet computers, to smartphones and PDAs, to storage devices, input devices, output devices, and more. Other electronic devices include game consoles, portable gaming devices, and music players. While these latter devices all include often powerful computational capabilities, their designs have been fine tuned for specific capabilities including graphics rendering speed and image resolution, processing speed, and audio fidelity. While some of these electronic devices are intended to be situated in offices or laboratories, many are designed to be readily portable. These latter devices can easily be slipped into a briefcase, messenger bag, or backpack, while others easily fit into pockets or can even be clipped to clothing. The devices can provide information, videos, and music, and can ease the tedium of routine tasks such as commuting. Whatever the intention, use, or purpose, there are electronic devices available to meet it.

One characteristic in common with all of the electronic devices is that people love to use their gadgets. The uses include consuming a wide variety of online content such as politics, news updates, sports scores, and other items of import, interest, amusement, and diversion. The uses often include consuming video streams such as TV programs, movies, and irresistible kitten and puppy videos. Other uses include keeping in touch with family, friends, coworkers, and other people through email, chat, social media, photos, and even telephony. The ways by which a user employs an electronic device to consume media or engage with others depend on the particular device. Smartphones are delightfully portable, enabling usage while a person is out and about. A smartphone can access the Internet; connect to news, information, and social media sites; enable online shopping; and support email, chatting, and calls, among myriad other uses. One disadvantage of the smartphone is that the smartphone display screen is relatively small. A tablet device offers much of the portability of the smartphone with the advantage of a larger display. The larger display makes interactions with others more enjoyable and greatly enhances media streaming. A laptop device is less portable than the smartphone or the tablet, but provides a still larger display. The laptop can access the Internet, interact with others, and engage in many other popular uses. The laptop offers the distinct advantage in that its more powerful processors are better suited to more complex uses such as creative activities, learning, and working.

### SUMMARY

Interest in health, wellness, nutrition, and fitness continues to become increasingly popular. People who participate in such interest areas do so for many good and varied reasons. Numerous people are interested in more than merely surviving, instead seeking activities, approaches, practices, and foods that help them to thrive. Recommendations from medical experts, meditation specialists, fitness trainers, and others are well known. These recommendations, which include encouragements to stop smoking, consume less or no alcohol, eat more fruits and vegetables, eat less overall, and exercise regularly are the common refrains that many people choose simply to ignore. Some recommendations border on the faddish such as eschewing carbohydrates for protein, or eating only once a day. Additional recommendations run to “biohacking”, where a person seeks to improve health or extend longevity through specific eating, exercise, or meditation practices, and, in some cases, the use of technology. Some individual choices are made by people based on availability of food types, cultural preferences, religious dictates, and so on. Other choices are made to improve health, to recover from surgery or injury, or to “turn over a new leaf” as far as lifestyle is concerned. Whatever the reason, a person can realize significant improvements to their health and general wellbeing by making well-founded choices.

The effectiveness of decisions that are made to improve health, fitness, wellness, nutrition, and so on can be determined by monitoring current health parameter values. The health parameter values include vital signs such as heart rate, blood pressure, and so on. The effectiveness of decisions for improvement can also be based on progress by the person toward a health goal. The effectiveness can be determined by measuring and monitoring a variety of health functionalities associated with the person over a period of time. The health monitoring can detect progress toward recovery from surgery or injury, weight loss, changes in body mass index (BMI), physical conditioning, and so on. The health monitoring can be based on collecting data from a variety of sensors that can be attached to, worn by, or otherwise accessible to a person whose health is being monitored. The sensors are used to collect health data which is used to detect health functionalities such as cardiac functionality, pulmonary functionality, three-dimensional motion, body temperature, and so on. The data collection can be based on the use of multiple sensors, including four sensors, five sensors, and so on. The four-sensor functionality of the person can be used to determine activity performance. The activity performance can include sports performance. The five-sensor functionality of the person can be used to determine non-activity performance. The non-activity performance can include sleep performance of the person. The sleep performance can include detecting sleep stages, nighttime conditions, and so on.

Intraoral electronic sensing enables health monitoring. Wireless connectivity is provided between a processor and a wireless transmitting device. The wireless transmitting device is embedded in an intraoral sensing interface for use in a person. A photoplethysmography (PPG) sensor is coupled to the wireless transmitting device. The PPG sensor is attached to the intraoral sensing interface, and the PPG sensor is used to detect cardiac functionality of the person. A breathing sensor is coupled to the wireless transmitting device. The breathing sensor is attached to the intraoral sensing interface, and the breathing sensor is used to detect pulmonary functionality of the person. An inertial measurement unit (IMU) sensor is used to detect three-dimensional motion of the person. The IMU can also be used to detect body position. A temperature sensor is coupled to the wireless transmitting device. The temperature sensor is attached to the intraoral sensing interface, and the temperature sensor is used to detect intraoral body temperature of the person. Health data about the person is provided to a receiving device, based on data from one or more of the PPG sensor, the breathing sensor, the IMU sensor, and the temperature sensor. The health data is provided using the wireless connectivity. The health data is used to monitor four-sensor health functionality of the person.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for intraoral electronic sensing for health monitoring.

FIG. 2 is a flow diagram for performance monitoring.

FIG. 3 is a system block diagram for intraoral electronic sensing.

FIG. 4 illustrates an intraoral device.

FIG. 5 shows a table of activity monitoring.

FIG. 6 is a table illustrating non-activity monitoring.

FIG. 7 is a diagram showing human teeth.

FIG. 8 is a system diagram for intraoral electronic sensing for health monitoring.

### DETAILED DESCRIPTION

In disclosed techniques, intraoral electronic sensing enables health monitoring. Data is collected from a plurality of sensors, where each sensor is used to detect a health functionality of a person. The health functionalities of the person can include cardiac functionality, pulmonary functionality, three-dimensional motion, body temperature, and so on. Wireless connectivity is provided between a processor and a wireless transmitting device. The wireless transmitting device is embedded in an intraoral sensing interface for use in a person. The intraoral sensing interface can include a device such as a bite plate, a splint, a mouthpiece, a retainer, dentures, and so on. A photoplethysmography (PPG) sensor is coupled to the wireless transmitting device. The PPG sensor is attached to the intraoral sensing interface, and the PPG sensor is used to detect cardiac functionality of the person. Cardiac function can include heart rate, heart rate variability, and the like. A breathing sensor is coupled to the wireless transmitting device. The breathing sensor is attached to the intraoral sensing interface, and the breathing sensor is used to detect pulmonary functionality of the person. Pulmonary function can include an inhale, an exhale, forced expiratory volume and capacity, etc. An inertial measurement unit (IMU) sensor is coupled to the wireless transmitting device. The IMU sensor is attached to the intraoral sensing interface, and the IMU sensor is used to detect three-dimensional motion of the person. The IMU can be used to detect body position. A temperature sensor is coupled to the wireless transmitting device. The temperature sensor is attached to the intraoral sensing interface, and the temperature sensor is used to detect intraoral body temperature of the person, which can be used to determine the core body temperature. The temperature can include a normal temperature, a low temperature (hypothermia), a high temperature (hyperthermia), and so on. Health data about the person is provided to a receiving device, based on data from one or more of the PPG sensor, the breathing sensor, the IMU sensor, and the temperature sensor. The health data is provided using the wireless connectivity.

Some embodiments include a processor-implemented method for health monitoring comprising: providing wireless connectivity between a processor and a wireless transmitting device, wherein the wireless transmitting device is embedded in an intraoral sensing interface for use in a person; coupling a photoplethysmography (PPG) sensor to the wireless transmitting device, wherein the PPG sensor is attached to the intraoral sensing interface, and wherein the PPG sensor is used to detect cardiac functionality of the person; coupling a breathing sensor to the wireless transmitting device, wherein the breathing sensor is attached to the intraoral sensing interface, and wherein the breathing sensor is used to detect pulmonary functionality of the person; coupling an inertial measurement unit (IMU) sensor to the wireless transmitting device, wherein the IMU sensor is attached to the intraoral sensing interface, and wherein the IMU sensor is used to detect three-dimensional motion of the person; coupling a temperature sensor to the wireless transmitting device, wherein the temperature sensor is attached to the intraoral sensing interface, and wherein the temperature sensor is used to detect intraoral body temperature of the person; and providing health data about the person to a receiving device, based on data from one or more of the PPG sensor, the breathing sensor, the IMU sensor, and the temperature sensor, wherein the health data is provided using the wireless connectivity.

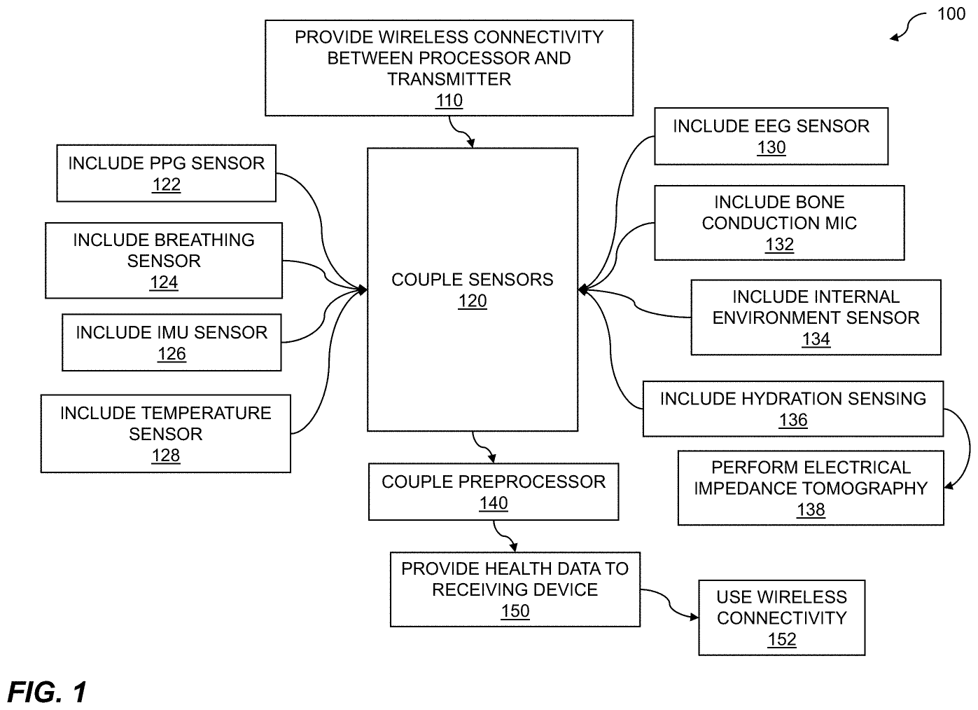

FIG. 1 is a flow diagram for intraoral electronic sensing for health monitoring. Data can be obtained from sensors, including augmented sensors, intelligent sensors, and so on, where the sensors can be attached to a device such as a prosthetic device that can be placed within the mouth of a user. The sensor data can be used to monitor health functionality of the user. The flow 100 includes providing wireless connectivity 110 between a processor and a wireless transmitting device. The wireless transmitting device is embedded in an intraoral sensing interface for use in a person. The wireless connectivity can be based on a variety of wireless communications techniques including standard wireless techniques such as the 802.11 family or “Wi-Fi™”, Bluetooth®, Bluetooth® Low Energy, Zigbee™, and so on. The wireless connectivity can be based on near field communication (NFC). The wireless connectivity can be based on near field magnetic induction (NFMI). The wireless connectivity can be provided as part of a wireless personal area network (WPAN). The processor can include a processor attached to the intraoral sensing device, a processor located adjacent to the intraoral sensing device, a remote processor, and the like. A processor such as a remote processor can include a desktop, laptop, or tablet computer; a smartphone; a PDA; etc.

The flow 100 includes coupling one or more sensors 120 to the wireless transmitting device. The one or more sensors can be attached to, embedded in, etc., the intraoral sensing interface. The flow 100 includes coupling a photoplethysmography (PPG) sensor 122 to the wireless transmitting device. The PPG sensor is attached to the intraoral sensing interface, and the PPG sensor is used to detect cardiac functionality of the person. Cardiac functionality of the person can include heart rate, heart rate variability, heart rate response, heart rate recovery, and so on. In embodiments, the PPG sensor can be located in an anchored position on the intraoral sensing interface. An anchored position can include a position adjacent to a specific tooth. In embodiments, the anchored position can be adjacent to an upper M1 molar of the person. In embodiments, the anchored position can be at a specified distance and angle from a particular tooth. The choice of anchored position of the PPG sensor can be made to improve efficacy of detecting cardiac functionality. In embodiments, the anchored position adjacent to an upper M1 molar can enable greater palatine artery monitoring.

The flow 100 includes coupling a breathing sensor 124 to the wireless transmitting device, wherein the breathing sensor is attached to the intraoral sensing interface, and wherein the breathing sensor is used to detect pulmonary functionality of the person. The breathing sensor can detect movement of air within the person's mouth, changes of pressure within the mouth, and so on. In embodiments, the breathing sensor detects inhales and/or exhales for the person. The breathing sensor can detect normal breathing of the person. The pulmonary functionality of the person can include spirometry parameters such as forced expiratory volume, forced expiratory capacity, and so on. In embodiments, the breathing sensor comprises a barometric pressure sensor. In other embodiments, the breathing sensor comprises a microphone. In some embodiments, the microphone is a bone-conduction microphone. In embodiments, the breathing sensor can detect a breathing disruption of the person. In other embodiments, an encapsulatory diaphragm can be attached to the sensing surface of a barometric sensor to enhance sensitivity of the sensor.

The flow 100 includes coupling an inertial measurement unit (IMU) sensor 126 to the wireless transmitting device. The IMU sensor is attached to the intraoral sensing interface, and the IMU sensor is used to detect three-dimensional motion of the person. The detected 3-D motion can include translation, rotation, acceleration, and so on. In embodiments, two or more IMUs can be attached to the intraoral sensing device. The IMUs can be attached at various locations within the mouth of the person. The locations can include occlusal locations (adjacent to teeth). In embodiments, the two or more IMUs are used for additional sports monitoring functions (discussed below and throughout). The flow 100 includes coupling a temperature sensor 128 to the wireless transmitting device, wherein the temperature sensor is attached to the intraoral sensing interface, and wherein the temperature sensor is used to detect the intraoral body temperature of the person. The intraoral body temperature can indicate or infer the body's core temperature. The intraoral body temperature can detect a normal body temperature, which is generally considered to be about 98.6° F. or 37° C. The intraoral body temperature can be detected and monitored on a continuing basis, such that body temperature trends are detected. The intraoral body temperature can also include a low temperature (hypothermia), and elevated temperature (hyperthermia), etc. The PPG, barometric, IMU, and temperature sensors can collect health data that can be used to monitor four-sensor health functionality of the person. The four-sensor functionality of the person can be used to determine activity performance such as sports performance.

The flow 100 further includes coupling an electroencephalogram (EEG) sensor 130 to the wireless transmitting device. The EEG sensor can be attached to the intraoral sensing interface, can be separate from the interface, and so on. In embodiments, the EEG sensor is used to detect brain activity of the person. The brain activity can include normal activity, abnormal activity such as activity during a seizure, and so on. In embodiments, the EEG sensor can provide data to augment the health data. The augmenting can include augmenting the four-sensor data discussed previously. The augmenting can include using primarily the EEG sensor data to understand brain activity. In embodiments, the health data that is augmented can be used to monitor five-sensor health functionality of the person. The five-sensor health functionality can be used for a variety of purposes. In embodiments, the five-sensor health functionality is used to determine non-activity performance. Non-activity performance can include rest performance of the person. In embodiments, the non-activity performance can include sleep performance of the person. The sleep performance can include normal sleep, sleep disorders, night conditions, etc. In embodiments, the sleep performance includes detecting sleep apnea. Sleep apnea can include stopping and starting of breathing while sleeping. Sleep apnea can result from the airway closing off while sleeping. Sleep apnea can manifest as loud snoring, gasping, and so on, and has many undesirable side effects which can include headaches, depression, stroke, or heart failure. In other embodiments, the sleep performance can identify sleep staging. Sleep staging can include transitioning from waking to sleep, non-rapid eye movement (NREM) sleep, rapid eye movement (REM) sleep, etc.

The flow 100 further includes coupling a bone-conduction microphone 132 to the wireless transmitting device. A microphone, either bone-conduction or non-bone-conduction, can augment or replace a breathing sensor. The bone-conduction microphone can detect sounds such as breathing sounds, speech, snoring, non-speech sounds, and so on. In embodiments, the bone-conduction microphone can be used to augment the health data of the person. For example, including breath sensing using a bone-conduction microphone can enable the distinction between nasal breathing and oral breathing. Furthermore, in a usage example, data from a barometric sensor and data from a bone-conduction microphone can be used to detect sleep apnea. Also, the bone-conduction microphone can detect bruxism, or “teeth grinding”, which can be quite harmful to the teeth. The flow 100 further includes coupling an internal environment sensor 134 for the person to the wireless transmitting device. The internal environment can include the internal environment of the mouth. In embodiments, the internal environment sensor can include one or more biometric sensors. The biometric sensors can detect “vital signs” associated with the person. In embodiments, the biometric sensors can include pH, oxygen, microbe, hormone, enzyme, blood pressure, jaw clenching force, and airflow sensors. The flow 100 further includes coupling a hydration sensor 136 to the wireless transmitting device. The hydration sensor can detect a normal level of hydration, dehydration, and the like. Data collected by the hydration sensor can be used for a variety of purposes. The flow 100 further includes performing electrical impedance tomography 138 on soft tissue in the mouth of the person. In other embodiments, data from the hydration sensor and the electrical impedance tomography enable mapping of the soft tissue in the mouth of the person.

The flow 100 further includes coupling an interface-embedded preprocessor 140 to the wireless transmitting device and one or more of the PPG sensor, the breathing sensor, the IMU sensor, and the temperature sensor. The preprocessor can be used to configure and control the sensors, to calibrate the sensors, to convert data collected from the sensors, and so on. The preprocessor can comprise a separate component from the other components associated with the intraoral sensing interface. In embodiments, the preprocessor and the wireless transmitting device can comprise an integrated electronic device. The integrated electronic device can include a chip, a field programmable gate array (FPGA), an application-specific integrated circuit (ASIC), etc. The flow 100 includes providing health data about the person 150 to a receiving device, based on data from one or more of the PPG sensor, the breathing sensor, the IMU sensor, and the temperature sensor. The providing health data can be accomplished using a variety of communications techniques. In the flow 100, the health data is provided using the wireless connectivity 152. The health data can include raw sensor data, calibrated data, converted data, and so on. The health data can include preprocessed data from the preprocessor. The health data that is provided can be analyzed. Analysis results can be rendered for the user. The rendered results can include progress toward a training goal, progress of recovery from surgery or injury, and so on. The health data can comprise input from various combinations of sensors. For example, certain monitoring modalities might only require one or two sensor inputs, while other monitoring modalities might require more than five sensor inputs.

Various embodiments of the flow 100 can be included in a computer program product embodied in a non-transitory computer readable medium that includes code executable by one or more processors.

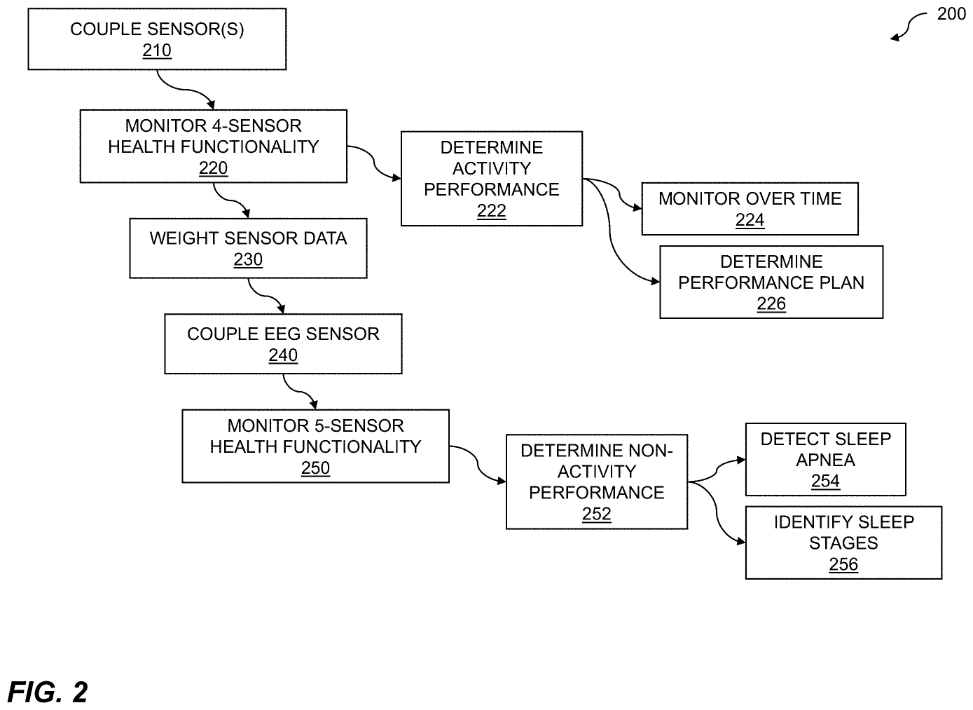

FIG. 2 is a flow diagram for performance monitoring. Sensors such as a photoplethysmography (PPG) sensor, a breathing sensor, inertial measurement units, a temperature sensor, biometric sensors, and so on, can be used to collect data from and process data for a person. The sensors and other components can be attached to a prosthetic device such as an intraoral device. The data collecting and processing enables monitoring of health functionality of person. The health functionality can be used to determine activity performance such as sports performance of the person, non-activity performance such as sleep performance, of the person, etc. Wireless connectivity is provided between a processor and a wireless transmitting device, wherein the wireless transmitting device is embedded in an intraoral sensing interface for use in a person. A photoplethysmography (PPG) sensor is coupled to the wireless transmitting device, wherein the PPG sensor is attached to the intraoral sensing interface, and wherein the PPG sensor is used to detect cardiac functionality of the person. A breathing sensor is coupled to the wireless transmitting device, wherein the breathing sensor is attached to the intraoral sensing interface, and wherein the breathing sensor is used to detect pulmonary functionality of the person. An inertial measurement unit (IMU) sensor is coupled to the wireless transmitting device, wherein the IMU sensor is attached to the intraoral sensing interface, and wherein the IMU sensor is used to detect three-dimensional motion of the person. A temperature sensor is coupled to the wireless transmitting device, wherein the temperature sensor is attached to the intraoral sensing interface, and wherein the temperature sensor is used to detect intraoral body temperature of the person. The intraoral body temperature of the person can indicate or reflect or correspond to a core body temperature measured intraorally. Health data about the person is provided to a receiving device, based on data from one or more of the PPG sensor, the breathing sensor, the IMU sensor, and the temperature sensor, wherein the health data is provided using the wireless connectivity.

A flow diagram for intraoral electronic sensing for health monitoring is shown. The flow 200 can include coupling one or more sensors 210 to a wireless transmitting device. Sensors of various types that serve a variety of purposes can be coupled to the transmitting device. In embodiments, the one or more sensors can include a photoplethysmography (PPG) sensor. The PPG sensor can be used to detect cardiac functionality of a person. The cardiac functionality of the person can include heart rate, heart rate variability, heart rate response, heart rate recovery, and so on. In embodiments, the one or more sensors can include a breathing sensor. The breathing sensor can be used to detect pulmonary functionality of the person. The pulmonary functionality of the person can include forced expiratory volume, forced expiratory capacity, breathing rate, breathing variability, and the like. In embodiments, an encapsulatory diaphragm can be attached to the sensing surface of a barometric-type breathing sensor to enhance sensitivity of the barometric-type sensor. In embodiments, the one or more sensors can include an inertial measurement unit (IMU) sensor. The IMU can be used to detect three-dimensional motion of the person, a position of the person, and so on. In embodiments, two IMUs can be attached in occlusal locations (e.g., adjacent to teeth) for additional sports monitoring functions. In embodiments, the one or more sensors can include a temperature sensor. The temperature sensor can be used to detect intraoral body temperature of the person. In other embodiments, the one or more sensors can include one or more biometric sensors. The biometric sensors can be used to detect heart rate, heart rate variability, respiratory rate, O2 saturation, breathing patterns, etc.

The flow 200 can include monitoring four-sensor health functionality 220. The four-sensor health functionality can include a PPG sensor, a breathing sensor, an IMU sensor, and a temperature sensor. Other sensor subsets and supersets can be employed to monitor health functionality. In the flow 200, the four-sensor functionality of the person can be used to determine activity performance 222. Activity performance can be associated with daily chores at home or on a farm, physical requirements of a job, and so on. In embodiments, the activity performance can include sports performance. Sports performance can be based on a level of performance, recovery from surgery or an injury, and the like. The flow 200 can further include monitoring the activity performance over time 224. Monitoring activity performance over time can be used to track improvements based on practice or training, to gauge recovery, to detect onset of a possible injury, to measure a decrease in performance, etc. The flow 200 can further include determining an activity performance plan 226, based on the monitoring. The performance plan can be designed and implemented to achieve peak performance for an event such as the World Cup, the Tour de France, or the Olympics. The performance plan can be designed to enhance recovery from surgery or injury.

The flow 200 further includes weighting the data 230 from one or more of the PPG sensor, the breathing sensor, the IMU sensor, and the temperature sensor to enable differentiated health functionality monitoring. The weighting can be used to bias or differentiate the monitoring to isolate one or more health functionalities, to favor one or more health functionalities, and so on. The one or more health functionalities can include cardiac functionality, pulmonary functionality, three-dimensional motion, body temperature, etc. The flow 200 further includes coupling an electroencephalogram (EEG) sensor 240 to the wireless transmitting device. The EEG sensor can be attached to a prosthetic device such as a mouthpiece, a bite plate, a retainer, and so on. In embodiments, the EEG sensor can used to detect brain activity of the person. The brain activity can include normal brain activity experienced while working, playing, engaging in sports, and so on. In embodiments, the EEG sensor, which can comprise a fifth sensor, can provide data to augment the health data. The EEG sensor can provide data associated abnormal brain activity such as can result from injury, a seizure, etc.

In the flow 200, the health data that is augmented is used to monitor five-sensor health functionality of the person 250. The five-sensor health functionality can include cardiac functionality, pulmonary functionality, 3-D motion, intraoral body temperature, brain activity, etc. In the flow 200, the five-sensor health functionality can be used to determine non-activity performance 252. Non-activity performance can be associated with standing or sitting quietly, remaining still, resting, and the like. In embodiments, the non-activity performance can include sleep performance of the person. The sleep performance can be used to determine sleep disorders. In the flow 200, the sleep performance can include detecting sleep apnea 254. The detecting sleep apnea can include detecting a severity of sleep apnea such as mild, moderate, severe, etc. In the flow 200, the sleep performance can identify sleep stages 256. The sleep stages can include non-rapid eye movement (NREM) sleep, rapid eye movement (REM) sleep, etc.

Various embodiments of the flow 200 can be included in a computer program product embodied in a non-transitory computer readable medium that includes code executable by one or more processors.

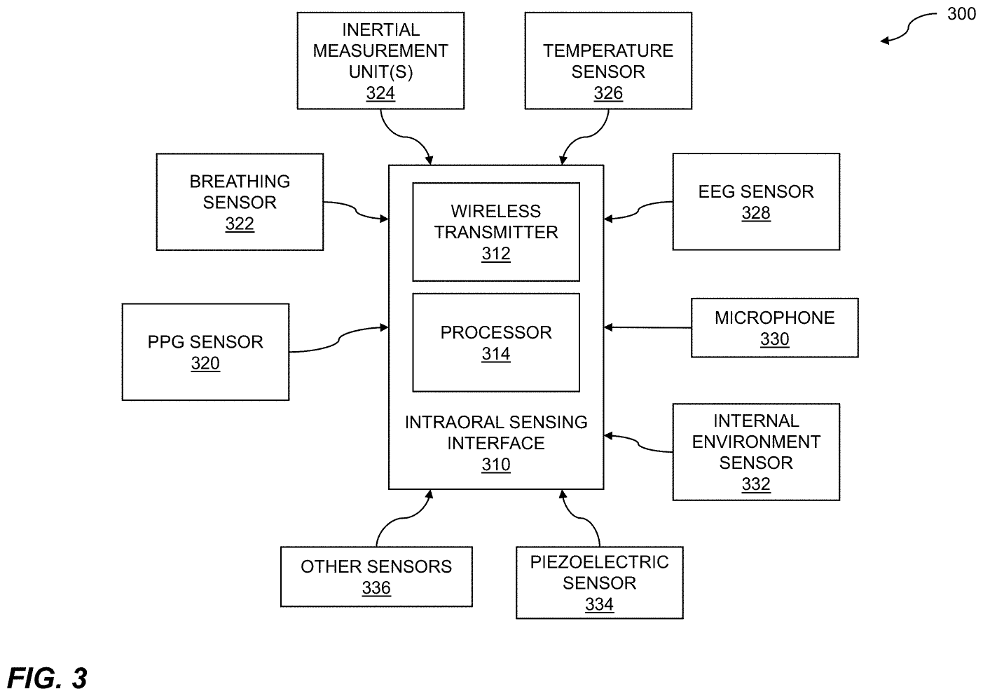

FIG. 3 is a system block diagram for intraoral electronic sensing. Discussed throughout, sensors of various types can be attached to an intraoral sensing interface. The sensors can be further coupled to a wireless transmitting device, which can provide data, collected from a person by using the one or more sensors, to a receiving device. Intraoral electronic sensing for health monitoring is used to monitor health functionality of a person. The health functionality of the person can be used to determine activity performance such as sports performance. The health functionality can be used to determine non-activity performance such as rest or sleep performance. Wireless connectivity is provided between a processor and a wireless transmitting device, wherein the wireless transmitting device is embedded in an intraoral sensing interface for use in a person. A photoplethysmography (PPG) sensor is coupled to the wireless transmitting device, wherein the PPG sensor is attached to the intraoral sensing interface, and wherein the PPG sensor is used to detect cardiac functionality of the person. A breathing sensor is coupled to the wireless transmitting device, wherein the breathing sensor is attached to the intraoral sensing interface, and wherein the breathing sensor is used to detect pulmonary functionality of the person. An inertial measurement unit (IMU) sensor is coupled to the wireless transmitting device, wherein the IMU sensor is attached to the intraoral sensing interface, and wherein the IMU sensor is used to detect three-dimensional motion of the person. A temperature sensor is coupled to the wireless transmitting device, wherein the temperature sensor is attached to the intraoral sensing interface, and wherein the temperature sensor is used to detect intraoral body temperature of the person. Health data about the person is provided to a receiving device, based on data from one or more of the PPG sensor, the breathing sensor, the IMU sensor, and the temperature sensor, wherein the health data is provided using the wireless connectivity.

The system block diagram 300 includes an intraoral sensing interface 310. The intraoral sensing interface can comprise a prosthetic device such as a “bite plate”, a mouthpiece, a retainer, and so on. The intraoral sensing interface is placed into the mouth of a person. Sensors of various types can be attached to the interface. The sensors can be used to collect data such as health data from the person wearing the interface. The intraoral sensing interface can include a wireless transmitting device 312. The wireless transmitting device can provide wireless connectivity between the intraoral sensing interface and a processor 314. The processor can include a processor embedded within the intraoral sensing interface, a processor coupled to the interface, and so on. The processor can include a processor chip, a processor core within a chip, etc. In embodiments, the processor can include an interface-embedded processor. The processor can be used to control sensors, to calibrate sensors, to collect sensor data, to convert sensor data, and so on.

One or more sensors can be coupled to the wireless transmitting device and can be attached to the intraoral sensing interface 310. A mouth can have no pigmentation. The lack of pigmentation can enhance or simplify sensing functions. In embodiments, a photoplethysmography (PPG) sensor 320 is coupled to the wireless transmitting device and the PPG sensor is attached to the intraoral sensing interface. The PPG sensor can be used to detect cardiac functionality of the person. The cardiac functionality can be based on data such as heart rate, heart rate variability, heart rate response, heart rate recovery, and so on. In embodiments, the PPG sensor is located in an anchored position on the intraoral sensing interface. The anchored position can be adjacent to a tooth. In embodiments, the anchored position can be adjacent to an upper M1 molar of the person. The anchored position can be chosen to improve arterial detection of a heartbeat, heart rate, etc. In embodiments, the anchored position adjacent to an upper M1 molar can enable greater palatine artery monitoring.

In embodiments, a breathing sensor 322 is coupled to the wireless transmitting device, wherein the breathing sensor is attached to the intraoral sensing interface. The breathing sensor can be used to detect pulmonary functionality of the person. In embodiments, the breathing sensor can detect inhales and/or exhales for the person. The pulmonary functionality of the person can include forced expiratory volume, forced expiratory capacity, and so on. The pulmonary functionality can be used to determine normal volume and capacity, diminished functionality such as during an asthma attack, etc. In embodiments, the breathing sensor can detect a breathing disruption of the person. The breathing disruption can be a result of a sleep disorder such as sleep apnea. In embodiments, an inertial measurement unit (IMU) sensor 324 is coupled to the wireless transmitting device, wherein the IMU sensor is attached to the intraoral sensing interface. The IMU sensor can be used to detect three-dimensional (3-D) motion of the person. The 3-D motion of the person can include movement of the head, movement of the body, etc. The 3-D motion can be based on head shakes, a blow to the head, tremors, and the like. In embodiments, the three-dimensional motion of the person can include a position of the person. In embodiments, more than one IMU is coupled to the wireless transmitting device.

In embodiments, a temperature sensor 326 is coupled to the wireless transmitting device, wherein the temperature sensor is attached to the intraoral sensing interface, and wherein the temperature sensor is used to detect intraoral body temperature of the person. The intraoral body temperature can include a temperature within a normal range of a healthy person. The intraoral body temperature can include an elevated temperature, where the elevated temperature can be the result of disease or illness, overheating due to excessive activity during hot weather, and the like. The PPE sensor, the breathing sensor, the IMU sensor, and the temperature sensor can provide data that can be used to monitor four-sensor health functionality of the person. The four-sensor health functionality of the person can be used to determine activity performance such as sports performance. The sports performance can be based on a target performance, current performance, healing performance, etc. Further embodiments include monitoring the activity performance over time. The monitoring performance over time can measure performance improvement, progress toward reaching a performance goal, etc. Embodiments further include determining an activity performance plan, based on the monitoring. Further embodiments include coupling an electroencephalogram (EEG) sensor 328 to the wireless transmitting device. The EEG sensor can be attached to the intraoral sensing interface to detect brain activity of the person. The brain activity can include normal brain activity, abnormal brain activity such as can occur during a seizure, and the like. In embodiments, the EEG sensor provides data to augment the health data, where the health data can be based on the four sensors previously discussed. In embodiments, the five-sensor health functionality can be used to determine non-activity performance, such as rest performance, sleep performance, and the like.

In embodiments, a microphone 330 is coupled to the wireless transmitting device, wherein the microphone is attached to the intraoral sensing interface. The microphone can include an audio microphone, a transducer, or other audio pickup device suitable for audio collection. The microphone can be operated normally on, normally off, etc. In embodiments, the microphone can be enabled based on the output from other sensors such as the IMU sensors. The microphone can capture audio data, speech data, and so on. In embodiments, the microphone can enable near-silent speech recognition. Further embodiments include coupling a bone-conduction microphone to the wireless transmitting device. The bone-conduction microphone can detect sound such as speech that can be conducted through the jaw, the skull, and so on. In embodiments, data from the bone-conduction microphone can be used to augment the health data of the person. In other embodiments, an internal environment sensor 332 for the person is coupled to the wireless transmitting device. The internal environment sensor can be attached to the intraoral sensing interface. The internal environment sensor can detect a variety of “vital signs”, or parameters associated with the person. In embodiments, the internal environment sensor can include one or more biometric sensors. The biometric sensors can collect biometric data associated with the person. In embodiments, the biometric sensors can include hydration, pH, oxygen, microbe, hormone, enzyme, blood pressure, jaw clenching force, and airflow sensors.

In embodiments, one or more piezoelectric sensors 334 are attached to the intraoral sensing interface. The one or more piezoelectric sensors can be attached to a closing surface of the interface such as over one or more teeth. The piezoelectric sensors can be used to sample biting pressure or force, grinding (lateral) pressure or force, and the like. Other sensors 336 can be attached to the intraoral sensing interface. The other sensors can include a tongue position sensor (TPS), a contact pressure sensor, and so on. In other embodiments, two IMUs can be attached to the interface at occlusal (next to the teeth) locations. The two IMUs can be used for further sports monitoring functions. Further embodiments include coupling a hydration sensor to the wireless transmitting device. The hydration sensor can be used to determine whether the person is sufficiently hydrated, is dehydrated, and so on. Further embodiments include performing electrical impedance tomography on soft tissue in the mouth of the person. Data from the hydration sensor and the electrical impedance tomography enable mapping of the soft tissue in the mouth of the person. The mapping can enable detection of normal tissue, infection, a tumor, etc.

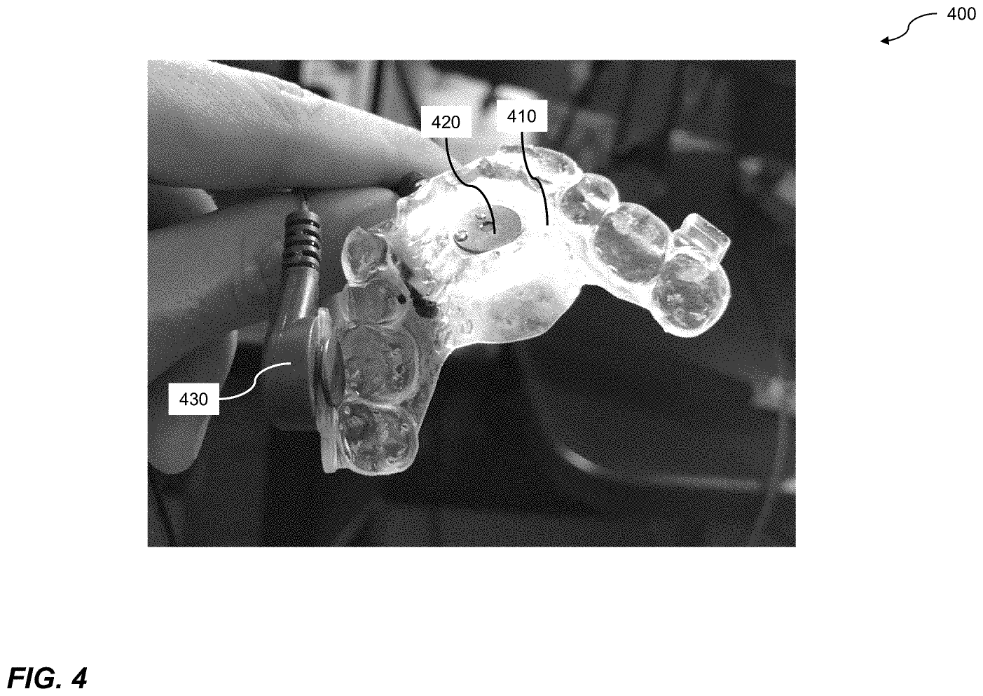

FIG. 4 illustrates an intraoral device. One or more sensors can be attached to an intraoral sensing interface. The intraoral sensing interface can use the sensors to collect health data associated with a person. The intraoral sensing interface can include a variety of oral prosthetics, devices, and so on. The intraoral sensing interface can include a mouthpiece, a plate such as a bite plate, a retainer, and the like. The intraoral sensing interface enables intraoral electronic sensing for health monitoring of a person. Wireless connectivity is provided between a processor and a wireless transmitting device, wherein the wireless transmitting device is embedded in an intraoral sensing interface for use in a person. A photoplethysmography (PPG) sensor is coupled to the wireless transmitting device, wherein the PPG sensor is attached to the intraoral sensing interface, and wherein the PPG sensor is used to detect cardiac functionality of the person. A breathing sensor is coupled to the wireless transmitting device, wherein the breathing sensor is attached to the intraoral sensing interface, and wherein the breathing sensor is used to detect pulmonary functionality of the person. An inertial measurement unit (IMU) sensor is coupled to the wireless transmitting device, wherein the IMU sensor is attached to the intraoral sensing interface, and wherein the IMU sensor is used to detect three-dimensional motion of the person. A temperature sensor is coupled to the wireless transmitting device, wherein the temperature sensor is attached to the intraoral sensing interface, and wherein the temperature sensor is used to detect intraoral body temperature of the person. Health data about the person is provided to a receiving device, based on data from one or more of the PPG sensor, the breathing sensor, the IMU sensor, and the temperature sensor, wherein the health data is provided using the wireless connectivity.

A configuration of an intraoral device is shown 400. The intraoral device can be worn by a person and can be used to capture sensor data associated with that person. The sensor data captured using the intraoral device can be provided to a receiving device. The receiving device can use the sensor data to monitor health functionality of the person. Discussed above and throughout, the sensor data, which can be captured by four sensors, five sensors, and so on, can be used to determine activity performance such as sports performance, non-activity performance such as rest performance or sleep performance, etc. The intraoral device can include a wearable device such as a retainer 410. The intraoral device can include a variety of components such as a wireless transmitting device, and sensors such as photoplethysmography (PPG), barometric, inertial measurement unit (IMU), and temperature sensors. The sensors can include a solid-state sensor, a micro-electro-mechanical system (MEMS), and so on. In embodiments, the sensors can include a microphone. The microphone can be used to collect audio data, speech data, and so on. The microphone can include an audio microphone, a transducer, or another component suitable for providing audio data to a data manipulation system. In embodiments, the microphone enables near-silent speech recognition. The output of the microphone can include an audio signal, where the audio signal can include an analog signal, a digital signal, etc. The microphone can include one or more usage states, where the usage states can include inactive, monitoring, etc. The microphone can be operated based on actions of a user of the microphone. In embodiments, the action of the microphone can be preprogrammed. The intraoral device can include a customized device such as a customized retainer, where the customized retainer is fitted to a particular user to ensure proper fit and to enable comfort of the user.

In addition to the four sensors (PPG, barometric, IMU, and temperature) mentioned previously, the intraoral device can further include an electroencephalogram (EEG) sensor. In embodiments, the electroencephalogram (EEG) sensor can be coupled to the wireless transmitting device associated with the intraoral sensing interface. The EEG can be mounted to the retainer 410. In embodiments, the EEG sensor can be used to detect brain activity of the person. The brain activity can include expected or normal activity, unusual or abnormal activity, and so on. In embodiments, the EEG sensor can provide data to augment the health data. Since the EEG sensor can comprise a fifth sensor coupled to the intraoral sensing device, in embodiments the health data that is augmented can be used to monitor five-sensor health functionality of the person. The five-sensor health functionality can be sued to determine performance such as non-activity performance. The non-activity performance can be associated with rest performance, sleep performance, detection of sleep stages, detection of sleep disorders, etc. The EEG sensor can include a signal source 420 and a reference 430 which can be in contact with the gingiva (gum) of a person. In embodiments, the EEG sensor can include one or more sets of tri-electrode intraoral contacts. The tri-electrode intraoral contacts can be embedded in the retainer, placed on the surface of the retainer, located adjacent to the retainer, and so on. In embodiments, the tri-electrode intraoral contacts can include a signal contact (e.g., 420), a reference contact (e.g., 430), and a ground contact (not shown). When two or more tri-electrode intraoral contacts are used, sets of tri-electrode intraoral contacts can utilize an electrically common ground contact. The EEG sensor can be placed in various positions relative to the intraoral device, electrical contacts, and so on. In embodiments, the EEG sensor can be placed in a contralateral configuration across the person's palate. The EEG sensor can be attached within the intraoral device, on a surface of the intraoral device, etc. In embodiments, the EEG sensor signal, or channel, electrode can contact the person's palate. Similar configurations can be arranged for the reference electrode. In embodiments, the reference electrode of the EEG sensor can contact the person's gumline. In embodiments, a signal electrode of the EEG sensor can contact the person's palate. In embodiments, a signal, or channel, electrode of the EEG sensor is placed contra-laterally with respect to a reference electrode of the EEG sensor.

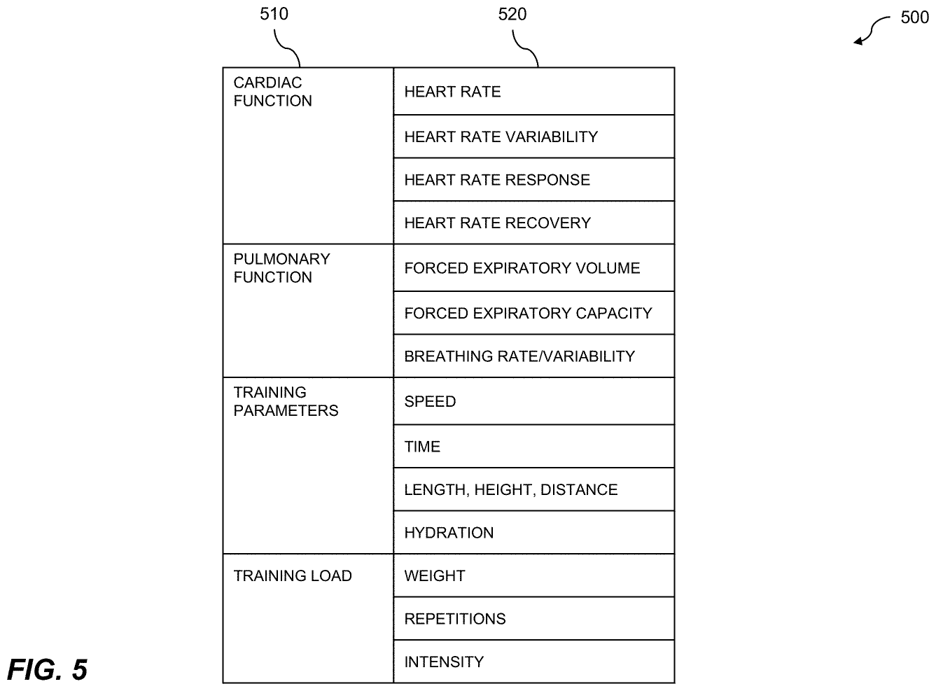

FIG. 5 shows a table of activity monitoring. An intraoral sensing interface can be coupled to a variety of sensors and can be used to monitor a wide range of health functionalities. The various health functionalities can be associated with a person. The one or more functionalities can be analyzed to determine a level of performance associated with the person. The performance can be based on physical activity, training, therapy, and the like. Health data can be collected from sensors such as a PPG sensor, a breathing sensor, an IMU sensor, a temperature sensor, a hydration sensor, and so on. In embodiments, the health data is used to monitor four-sensor health functionality of the person. The health functionality can be used to determine training progress, presence of injury, rehabilitation therapy effectiveness, and the like. In other embodiments, the four-sensor functionality of the person can be used to determine activity performance. The activity performance can be associated with sports performance, physical task performance, etc.

Activity monitoring is enabled by intraoral electronic sensing for health monitoring. Wireless connectivity is provided between a processor and a wireless transmitting device, wherein the wireless transmitting device is embedded in an intraoral sensing interface for use in a person. A photoplethysmography (PPG) sensor is coupled to the wireless transmitting device, wherein the PPG sensor is attached to the intraoral sensing interface, and wherein the PPG sensor is used to detect cardiac functionality of the person. A breathing sensor is coupled to the wireless transmitting device, wherein the breathing sensor is attached to the intraoral sensing interface, and wherein the breathing sensor is used to detect pulmonary functionality of the person. An inertial measurement unit (IMU) sensor is coupled to the wireless transmitting device, wherein the IMU sensor is attached to the intraoral sensing interface, and wherein the IMU sensor is used to detect three-dimensional motion of the person. A temperature sensor is coupled to the wireless transmitting device, wherein the temperature sensor is attached to the intraoral sensing interface, and wherein the temperature sensor is used to detect intraoral body temperature of the person. Health data about the person is provided to a receiving device, based on data from one or more of the PPG sensor, the breathing sensor, the IMU sensor, and the temperature sensor, wherein the health data is provided using the wireless connectivity.

The intraoral sensing interface can be used to monitor activity performance by collecting sensor data from the variety of sensors. Further embodiments can include monitoring the activity performance over time. The period of time over which activity performance can be monitored can include before and after the activity; over a course of hours, days, weeks, or months; etc. The period of time can include a sports season. Further embodiments can include determining an activity performance plan, based on the monitoring. The activity performance plan can be designed to enable peak performance for a particular event such as the Olympics, the Tour de France, the World Cup, the Superbowl, the World Series, etc. Further embodiments include weighting the data from one or more of the PPG sensor, the breathing sensor, the IMU sensor, and the temperature sensor to enable differentiated health functionality monitoring.

A table for activity monitoring 500 is shown. The activity monitoring can be based on four-sensor health functionality of the person, where the four-sensor health functionality can be based on a PPG sensor, a breathing sensor, an IMU sensor, and a temperature sensor. In embodiments, a hydration sensor is included. In embodiments, an EEG sensor can also be used for activity monitoring. Further embodiments can include coupling an electroencephalogram (EEG) sensor to the wireless transmitting device. The table 500 shows examples of functions and parameters associated with activity performance 510. The table 500 further shows types of data collected by the sensors 520 that are associated with the functions and parameters. In embodiments, the activity performance can include cardiac function of the person. The cardiac function of the person can be based on heart rate, heart rate variability, heart rate response, heart rate recovery, and so on. The activity performance can include pulmonary function. The pulmonary function can be based on forced expiratory volume, forced expiratory capacity, breathing rate and variability, and the like. The activity performance can include training parameters, where the training parameters can be designed for the person. The training parameters can include speed, time, length and height, distance covered, etc. The activity performance can further include training load. The training load can include weight lifted or carried, number of repetitions, intensity of a training session, and the like.

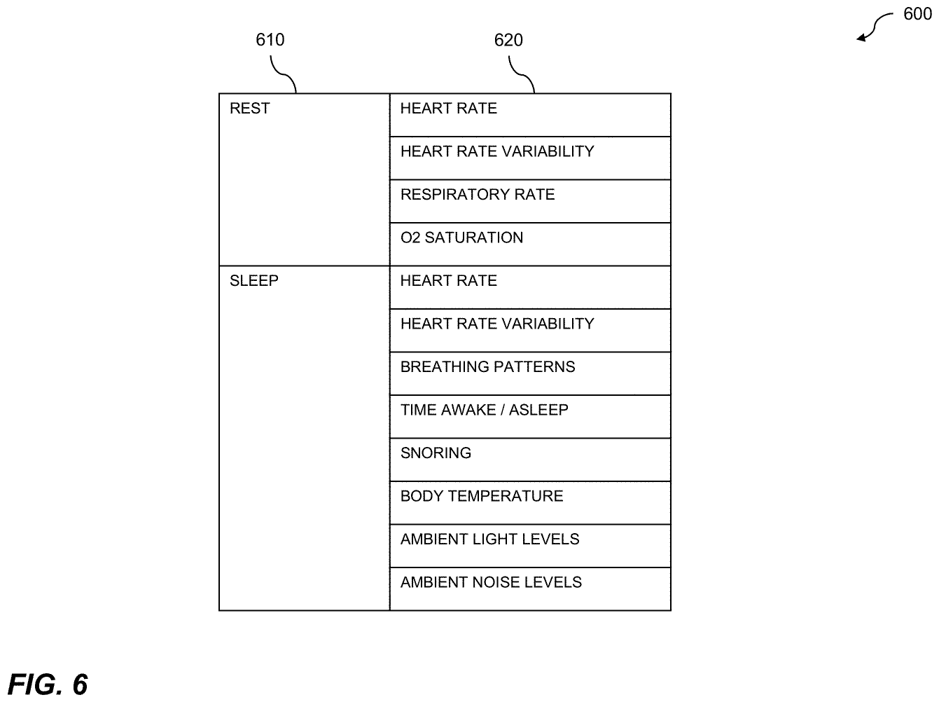

FIG. 6 is a table illustrating non-activity monitoring. Discussed above and throughout, an intraoral sensing interface can be used to monitor a variety of health functionalities associated with a person. The one or more of the functionalities can be considered to determine a level of performance associated with the person. In embodiments, health functionality can be used to determine non-activity performance. Non-activity performance, in contrast to activity performance, can include rest performance, sleep performance, and so on. Non-activity monitoring is enabled by intraoral electronic sensing for health monitoring. Wireless connectivity is provided between a processor and a wireless transmitting device, wherein the wireless transmitting device is embedded in an intraoral sensing interface for use in a person. A photoplethysmography (PPG) sensor is coupled to the wireless transmitting device, wherein the PPG sensor is attached to the intraoral sensing interface, and wherein the PPG sensor is used to detect cardiac functionality of the person. A breathing sensor is coupled to the wireless transmitting device, wherein the breathing sensor is attached to the intraoral sensing interface, and wherein the breathing sensor is used to detect pulmonary functionality of the person. An inertial measurement unit (IMU) sensor is coupled to the wireless transmitting device, wherein the IMU sensor is attached to the intraoral sensing interface, and wherein the IMU sensor is used to detect three-dimensional motion of the person. A temperature sensor is coupled to the wireless transmitting device, wherein the temperature sensor is attached to the intraoral sensing interface, and wherein the temperature sensor is used to detect intraoral body temperature of the person. Health data about the person is provided to a receiving device, based on data from one or more of the PPG sensor, the breathing sensor, the IMU sensor, and the temperature sensor, wherein the health data is provided using the wireless connectivity.

A table for non-activity monitoring is illustrated. The non-activity monitoring can be based on four-sensor health functionality of the person, where the four-sensor health functionality can be based on a PPG sensor, a breathing sensor, an IMU sensor, and a temperature sensor. Further embodiments can include coupling an electroencephalogram (EEG) sensor to the wireless transmitting device. The EEG sensor, which can detect brain activity in a person, can be used to provide data to augment the health data obtained using the four sensors previously discussed. The health data that is augmented can be used to monitor five-sensor health functionality of the person. In embodiments, the five-sensor health functionality can be used to determine non-activity performance. The table 600 shows examples of non-activity performance 610. The table 600 further shows parameters detected by the sensors 620. In embodiments, the non-activity performance can include rest performance of the person. The rest performance of the person can be based on heart rate, heart rate variability, respiratory rate, O2 saturation, and so on. The rest performance can be used to determine a recovery rate from an activity, from injury, and so on. In other embodiments, the non-activity performance can include sleep performance of the person. The sleep performance can be based on heart rate, heart rate variability, breathing patterns, time awake or asleep, body temperature, ambient light levels, ambient noise levels, and so on. The sleep performance can be used to determine effectiveness of sleep, to identify sleep disorders, and so on. In embodiments, the sleep performance can include detecting sleep apnea. Sleep apnea can cause sleep to be less effective and efficient, can cause serious health problems, etc. In other embodiments, the sleep performance can identify sleep stages. Stages of sleep can include a plurality of sleep stages associated with non-rapid eye movement (NREM) sleep and rapid eye movement (REM) sleep.

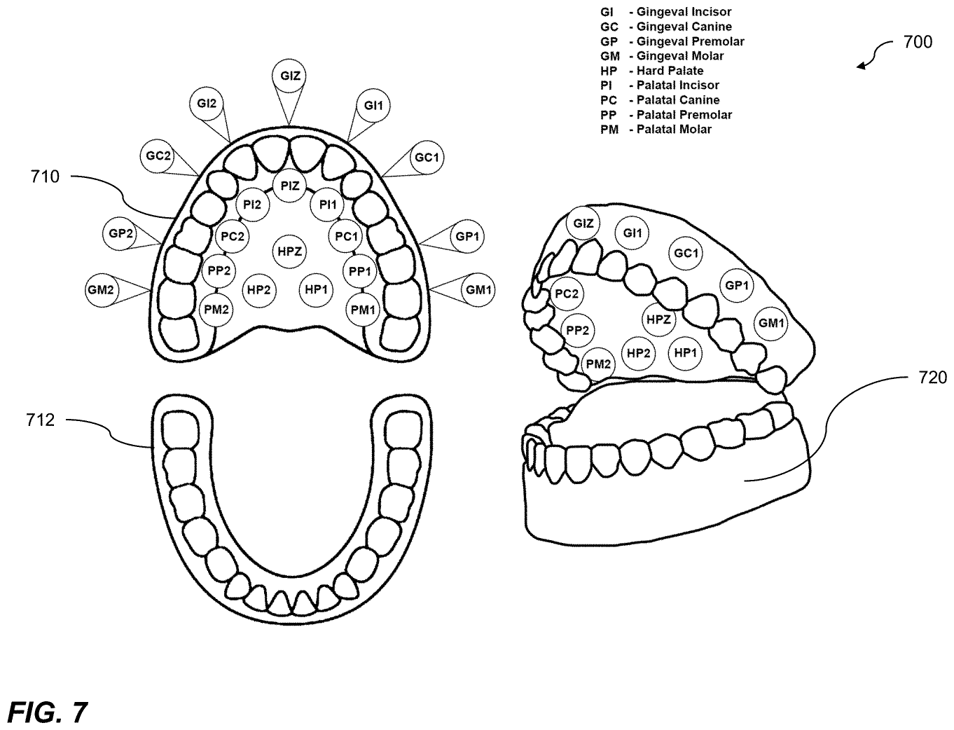

FIG. 7 is a diagram showing human teeth. An intraoral sensing interface can use one or more sensors to detect various types of data such as health data. The health data can be associated with health functionality of a person. The sensors can be attached to the intraoral sensing interface, and the sensors can further be coupled to a wireless transmitting device which can be connected to a processor. The intraoral sensing interface can comprise a prosthesis such as a retainer, where the retainer can be worn within the mouth of the person. The retainer can be in contact with one or more teeth within the mouth of the person. The intraoral sensing interface enables health monitoring of the person. The diagram 700 shows two views of human teeth. A “top down” view of human teeth includes upper teeth 710 and lower teeth 712. The tops, sides, fronts, backs, contact points, and so on of the teeth can be identified. Sides of the upper teeth can include gingival (adjacent to gum) sides and palatal (adjacent to palate) sides. The sides of particular teeth can be labeled and can include a gingival incisor (GI), a gingival canine (GC), a gingival premolar (GP), and so on. The sides of particular teeth can further include a palatal incisor (PI), a palatal canine (PC), a palatal premolar (PP), and a palatal molar (PM). The palate can also be labeled as hard palate (HP). The lower teeth can also be labeled by their gingival sides. The inside surfaces of the lower teeth are labeled as lingual. A side view of upper and lower teeth is also shown 720. The various sensors and other components of the intraoral interface can be located in contact with one or more teeth, adjacent to one or more teeth, in contact with fleshy tissue within the mouth such as the gingiva (gums), in contact with the palate, etc. A prosthetic such as a retainer, biteplate, etc., can include an intraoral sensing interface. Sensors associated with the intraoral sensing interface can be used to detect tongue movement, biting pressure, etc. The sensors can further detect cardiac function, pulmonary function, three-dimensional motion, body temperature, brain activity, and the like.

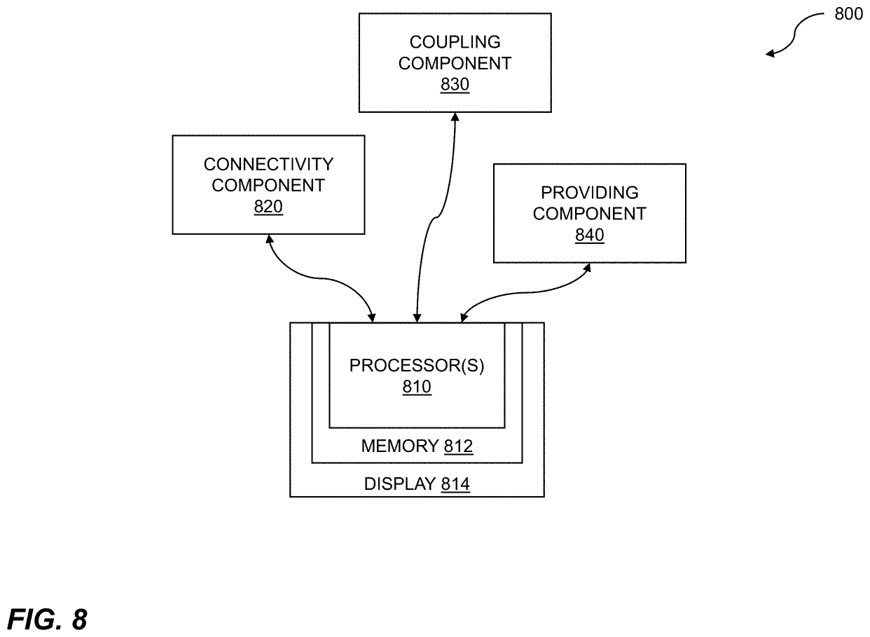

FIG. 8 is a system diagram for intraoral electronic sensing for health monitoring. Intraoral electronic sensing can be used for data manipulation. Electronic intraoral sensors can be elements of an intraoral sensing interface that can be used by a person. Wireless connectivity is provided between the intraoral sensing interface and a processor which can process sensor data for the health monitoring. The sensors can include sensors remote to the processor such as sensors within a user's mouth, sensors adjacent to the user, and so on. In embodiments, the sensors can include a photoplethysmography (PPG) sensor, a breathing sensor, an inertial measurement unit (IMU) sensor, a temperature sensor, and so on. The sensors can be used to measure or detect four-sensor health functionality of the person. A further electroencephalogram (EEG) sensor can be coupled to the wireless transmitting device. The EEG sensor can provide data to augment the health data, where the health data that is augmented can be used to monitor five-sensor health functionality of the person. The five-sensor health functionality can used to determine non-activity performance such as sleep performance of the person. The health monitoring can further be accomplished using a prosthetic intraoral device such as a retainer, where the retainer can further be used to measure tongue position, pressure, and the like.

Wireless connectivity is provided between a processor and a wireless transmitting device, wherein the wireless transmitting device is embedded in an intraoral sensing interface for use in a person. A photoplethysmography (PPG) sensor is coupled to the wireless transmitting device, wherein the PPG sensor is attached to the intraoral sensing interface, and wherein the PPG sensor is used to detect cardiac functionality of the person. A breathing sensor is coupled to the wireless transmitting device, wherein the breathing sensor is attached to the intraoral sensing interface, and wherein the breathing sensor is used to detect pulmonary functionality of the person. An inertial measurement unit (IMU) sensor is coupled to the wireless transmitting device, wherein the IMU sensor is attached to the intraoral sensing interface, and wherein the IMU sensor is used to detect three-dimensional motion of the person. A temperature sensor is coupled to the wireless transmitting device, wherein the temperature sensor is attached to the intraoral sensing interface, and wherein the temperature sensor is used to detect intraoral body temperature of the person. Health data about the person is provided to a receiving device, based on data from one or more of the PPG sensor, the breathing sensor, the IMU sensor, and the temperature sensor, wherein the health data is provided using the wireless connectivity. The health data is used to monitor four-sensor health functionality of the person.

The system 800 can include one or more processors 810 and a memory 812 which stores instructions. The memory 812 is coupled to the one or more processors 810, wherein the one or more processors 810 can execute instructions stored in the memory 812. The memory 812 can be used for storing instructions, runtime libraries, data manipulation routines, error codes or handling routines, and so on. The memory can further be used for storing sensor calibration data, sensor data conversion tables, and the like. Information such as sensor data can be shown on a display 814 connected to the one or more processors 810. The display can comprise a television monitor, a projector, a computer monitor (including a laptop screen, a tablet screen, a netbook screen, and the like), a smartphone display, a mobile device, or another electronic display. The system 800 can include a connectivity component 820. The connectivity component 820 can be used for providing wireless connectivity and communication between a processor and a wireless transmitting device, wherein the wireless transmitting device is embedded in an intraoral sensing interface for use in a person. The processor, which can be outside the mouth, can include a computer such as a laptop or desktop computer, a smartphone, a PDA, a tablet, and so on.

The communicating can be accomplished using wireless techniques, which generally includes the use of electromagnetically-based connectivity, but can also include other technique such as sound-based techniques using air, tissue, or bone as a medium. In embodiments, a sensor such as an EEG sensor can include one or more sets of tri-electrode intraoral contacts. The tri-electrode contacts can be associated with a wire, a serpentine wire, and the like. In embodiments, the tri-electrode intraoral contacts can include a signal contact, a reference contact, and a ground contact. The ground contact can be based on an electrically common ground contact. The wireless connectivity can be based on wireless communications standards and techniques such as 802.11 Wi-Fi™, Bluetooth™, near field communication (NFC), near field magnetic induction (NFMI), ZigBee™, a wireless personal area network (WPAN), and so on. The wireless connectivity can include bidirectional communications capabilities. In embodiments, the preprocessor and the wireless transmitting device comprise an integrated electronic device.

The system 800 can include a coupling component 830. The coupling component 830 can be used for coupling one or more sensors to the wireless transmitting device. In embodiments, the coupling component can couple a photoplethysmography (PPG) sensor to the wireless transmitting device. The PPG sensor can be attached to an interface such as the intraoral sensing interface. The PPG sensor can be used for a variety of sensing purposes. In embodiments, the PPG sensor can be used to detect cardiac functionality of the person. In embodiments, the coupling component can couple a breathing sensor to the wireless transmitting device. The breathing sensor can be attached to the intraoral sensing interface for various sensing techniques. In embodiments, the breathing sensor can be used to detect pulmonary functionality of the person. The breathing sensor can be used to detect pressure and duration of an inhale, pressure and duration of an exhale, respiratory rate, and the like. In embodiments, an encapsulatory diaphragm can be attached to a sensing surface of the breathing sensor. The encapsulatory diaphragm can enhance sensitivity of the breathing sensor.

In embodiments, the coupling component can couple an inertial measurement unit (IMU) sensor to the wireless transmitting device. The IMU sensor is attached to the intraoral sensing interface, and is used to detect three-dimensional motion of the person. The three-dimensional motion can include position, rotation, acceleration, etc. In other embodiments, the coupling component can couple a temperature sensor to the wireless transmitting device. The temperature sensor can be attached to the intraoral sensing interface and can be used to detect the intraoral body temperature of the person. The intraoral body temperature can be based on a person at rest, a person exercising, an ill person, and so on. In further embodiments, the coupling component can couple an electroencephalogram (EEG) sensor to the wireless transmitting device. The EEG, which measures electrical activity in the brain, can be used to detect normal or baseline function of the brain, abnormal function, etc. In embodiments, the EEG sensor can provide data to augment the health data. Other sensors can be coupled to the wireless transmitting device. In embodiments, one or more piezoelectric sensors can be coupled to the wireless transmitting device. The piezoelectric sensors can be attached to a closing surface of the interface, where the closing surface can be located over one or more teeth. The piezoelectric sensors can be used to measure biting pressure, patterns, alignment, etc.

The system 800 includes a providing component 840. The providing component 840 can be used for providing health data about the person to a receiving device. The data that is presented can be based on data from one or more sensors. The sensors can include one or more of the PPG sensor, the breathing sensor, the IMU sensor, and the temperature sensor, wherein the health data is provided using the wireless connectivity. In embodiments, the health data can be used to monitor four-sensor health functionality of the person. The health functionality of the person can include checking vital statistics, monitoring the person while he or she is engaged in activities such as sports activities, monitoring the person at rest, etc. The four-sensor functionality of the person can be used to determine activity performance. In embodiments, the activity performance can include sports performance. Discussed above and throughout, the sensors can further include one or more EEG sensors to monitor brain activity. An EEG sensor provides a fifth sensor which enables monitoring of five-sensor health functionality of the person. In embodiments, the five-sensor health functionality can be used to determine non-activity performance. Non-activity performance can include resting performance of a person, sleeping performance, and so on. In embodiments, the non-activity performance can include sleep performance of the person. The sleep performance can be based on data associated with sleep stages, sleep disorders such as sleep apnea, etc.

Disclosed embodiments can include a computer program product embodied in a non-transitory computer readable medium for health monitoring, the computer program product comprising code which causes one or more processors to perform operations of: providing wireless connectivity between a processor and a wireless transmitting device, wherein the wireless transmitting device is embedded in an intraoral sensing interface for use in a person; coupling a photoplethysmography (PPG) sensor to the wireless transmitting device, wherein the PPG sensor is attached to the intraoral sensing interface, and wherein the PPG sensor is used to detect cardiac functionality of the person; coupling a breathing sensor to the wireless transmitting device, wherein the breathing sensor is attached to the intraoral sensing interface, and wherein the breathing sensor is used to detect pulmonary functionality of the person; coupling an inertial measurement unit (IMU) sensor to the wireless transmitting device, wherein the IMU sensor is attached to the intraoral sensing interface, and wherein the IMU sensor is used to detect three-dimensional motion of the person; coupling a temperature sensor to the wireless transmitting device, wherein the temperature sensor is attached to the intraoral sensing interface, and wherein the temperature sensor is used to detect intraoral body temperature of the person; and providing health data about the person to a receiving device, based on data from one or more of the PPG sensor, the breathing sensor, the IMU sensor, and the temperature sensor, wherein the health data is provided using the wireless connectivity.

Each of the above methods may be executed on one or more processors on one or more computer systems. Embodiments may include various forms of distributed computing, client/server computing, and cloud-based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general-purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are limited neither to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A processor-implemented method for health monitoring comprising:
providing wireless connectivity between a processor and a wireless transmitting device, wherein the wireless transmitting device is embedded in an intraoral sensing interface for use in a person;
coupling a photoplethysmography (PPG) sensor to the wireless transmitting device, wherein the PPG sensor is attached to the intraoral sensing interface, and wherein the PPG sensor is used to detect cardiac functionality of the person;
coupling a breathing sensor to the wireless transmitting device, wherein the breathing sensor is attached to the intraoral sensing interface, and wherein the breathing sensor is used to detect pulmonary functionality of the person;
coupling an inertial measurement unit (IMU) sensor to the wireless transmitting device, wherein the IMU sensor is attached to the intraoral sensing interface, and wherein the IMU sensor is used to detect three-dimensional motion of the person;
coupling a temperature sensor to the wireless transmitting device, wherein the temperature sensor is attached to the intraoral sensing interface, and wherein the temperature sensor is used to detect intraoral body temperature of the person; and
providing health data about the person to a receiving device, based on data from one or more of the PPG sensor, the breathing sensor, the IMU sensor, and the temperature sensor, wherein the health data is provided using the wireless connectivity.

2. The method of claim 1 wherein the health data is used to monitor four-sensor health functionality of the person.

3. The method of claim 2 wherein the four-sensor functionality of the person is used to determine activity performance.

4. The method of claim 3 further comprising monitoring the activity performance over time.

5. The method of claim 4 further comprising determining an activity performance plan, based on the monitoring.

6. The method of claim 3 further comprising weighting the data from one or more of the PPG sensor, the breathing sensor, the IMU sensor, and the temperature sensor to enable differentiated health functionality monitoring.

7. The method of claim 1 further comprising coupling an electroencephalogram (EEG) sensor to the wireless transmitting device.

8. The method of claim 7 wherein the EEG sensor provides data to augment the health data.

9. The method of claim 8 wherein the health data that is augmented is used to monitor five-sensor health functionality of the person.

10. The method of claim 9 wherein the five-sensor health functionality is used to determine non-activity performance.

11. The method of claim 10 wherein the non-activity performance includes sleep performance of the person.

12. The method of claim 11 wherein the sleep performance includes detecting nighttime conditions.

13. The method of claim 11 wherein the sleep performance identifies sleep stages.

14. The method of claim 7 wherein the EEG sensor comprises one or more sets of tri-electrode intraoral contacts.

15. The method of claim 14 wherein the tri-electrode intraoral contacts comprise a signal contact, a reference contact, and a ground contact.

16. The method of claim 14 wherein the one or more sets of tri-electrode intraoral contacts utilize an electrically common ground contact.

17. The method of claim 7 wherein the EEG sensor is placed in a contralateral configuration across a person's palate.

18. The method of claim 1 further comprising coupling an interface-embedded preprocessor to the wireless transmitting device and one or more of the PPG sensor, the breathing sensor, the IMU sensor, and the temperature sensor.

19. The method of claim 18 wherein the preprocessor and the wireless transmitting device comprise an integrated electronic device.

20. The method of claim 1 wherein the breathing sensor detects inhales and/or exhales for the person.

21. The method of claim 1 wherein the breathing sensor comprises a barometric pressure sensor.

22. The method of claim 1 wherein the breathing sensor comprises a microphone.

23. The method of claim 22 wherein the microphone is a bone-conduction microphone.

24. The method of claim 1 wherein the PPG sensor is located in an anchored position on the intraoral sensing interface.

25. The method of claim 24 wherein the anchored position is adjacent to an upper M1 molar of the person.

26. A computer program product embodied in a non-transitory computer readable medium for health monitoring, the computer program product comprising code which causes one or more processors to perform operations of:
providing wireless connectivity between a processor and a wireless transmitting device, wherein the wireless transmitting device is embedded in an intraoral sensing interface for use in a person;
coupling a photoplethysmography (PPG) sensor to the wireless transmitting device, wherein the PPG sensor is attached to the intraoral sensing interface, and wherein the PPG sensor is used to detect cardiac functionality of the person;
coupling a breathing sensor to the wireless transmitting device, wherein the breathing sensor is attached to the intraoral sensing interface, and wherein the breathing sensor is used to detect pulmonary functionality of the person;
coupling an inertial measurement unit (IMU) sensor to the wireless transmitting device, wherein the IMU sensor is attached to the intraoral sensing interface, and wherein the IMU sensor is used to detect three-dimensional motion of the person;
coupling a temperature sensor to the wireless transmitting device, wherein the temperature sensor is attached to the intraoral sensing interface, and wherein the temperature sensor is used to detect intraoral body temperature of the person; and
providing health data about the person to a receiving device, based on data from one or more of the PPG sensor, the breathing sensor, the IMU sensor, and the temperature sensor, wherein the health data is provided using the wireless connectivity.

27. A computer system for health monitoring comprising:
a memory which stores instructions;
one or more processors coupled to the memory wherein the one or more processors, when executing the instructions which are stored, are configured to:
provide wireless connectivity between a processor and a wireless transmitting device, wherein the wireless transmitting device is embedded in an intraoral sensing interface for use in a person;
couple a photoplethysmography (PPG) sensor to the wireless transmitting device, wherein the PPG sensor is attached to the intraoral sensing interface, and wherein the PPG sensor is used to detect cardiac functionality of the person;
couple a breathing sensor to the wireless transmitting device, wherein the breathing sensor is attached to the intraoral sensing interface, and wherein the breathing sensor is used to detect pulmonary functionality of the person;
couple an inertial measurement unit (IMU) sensor to the wireless transmitting device, wherein the IMU sensor is attached to the intraoral sensing interface, and wherein the IMU sensor is used to detect three-dimensional motion of the person;
couple a temperature sensor to the wireless transmitting device, wherein the temperature sensor is attached to the intraoral sensing interface, and wherein the temperature sensor is used to detect intraoral body temperature of the person; and
provide health data about the person to a receiving device, based on data from one or more of the PPG sensor, the breathing sensor, the IMU sensor, and the temperature sensor, wherein the health data is provided using the wireless connectivity.
